(19) United States
(12) Patent Application Publication
(10) Pub. No.: US 2008/0269071 A1
(43) Pub. Date: Oct. 30, 2008

(54) PSEUDOPTEROSIN-PRODUCING BACTERIA AND METHODS OF USE

(76) Inventors: Sutaporn BUNYAJETPONG, Charlottetown (CA); Lory Z. Santiago-Vazquez, Houston, TX (US); Russell Kerr, Charlottetown (CA)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 12/111,597

(22) Filed: Apr. 29, 2008

Related U.S. Application Data
(60) Provisional application No. 60/914,856, filed on Apr. 30, 2007.

(51) Int. Cl. C40B 40/02; C12N 1/20; C12P 1/00

(52) U.S. Cl. 506/14; 435/252.34; 435/170

(57) ABSTRACT

Clonal strains of bacteria derived from Pseudopterogorgia elisabethae are capable of making pseudopterosins in in vitro cultures without requiring the presence of other bacteria, algae, or animal cells that are normally present in P. elisabethae.

### CROSS-REFERENCE TO RELATED APPLICATIONS

The present application claims the priority of U.S. provisional patent application No. 60/914,856, filed on Apr. 30, 2007.

### STATEMENT AS TO FEDERALLY-SPONSORED RESEARCH

Not applicable.

### FIELD OF THE INVENTION

The invention relates to the fields of marine microbiology, natural products chemistry, and terpene production methods. More particularly, the invention relates to pseudopterosin-producing bacteria and methods of using such bacteria to produce pseudopterosin.

### BACKGROUND OF THE INVENTION

A number of biologically active compounds with potential commercial applications have been derived from marine organisms. Among these, the pseudopterosins are a group of diterpene glycosides isolated from the Caribbean sea whip, Pseudopterogorgia elisabethae. The pseudopterosins represent an important structural class of anti-inflammatory and analgesic metabolites, and exhibit superior analgesic activity compared to industrial standards such as indomethacin. Currently, pseudopterosins for use in commercial products and clinical trials are obtained by extraction of P. elisabethae harvested from coral reefs.

### SUMMARY

The invention is based on the surprising discovery that isolated clonal strains of bacteria derived from Pseudopterogorgia elisabethae are capable of making pseudopterosins in in vitro cultures without requiring the presence of other bacteria, algae, or animal cells that are normally present in P. elisabethae. This is significant because it is generally believed that natural products such as the pseudopterosins are produced to help promote the survival of the producing organism in a complex ecological environment and thus the presence of a variety of organisms is required for the production of such natural products. See e.g., Angell et al., 2006. Chem. Biol. 13:1349-59 (two bacteria of marine origin required to produce pyocyanin).

In making the invention, a zooxanthellae-enriched fraction was isolated from a sample of P. elisabethae collected from the waters near Bimini, The Bahamas. This fraction was inoculated into culture medium to produce a mixed bacterial culture. After several subcultures, the resulting culture was diluted and plated on solid agar medium. Several colonies that grew on the solid medium were shown to produce pseudopterosins upon subsequent culture in liquid medium.

This discovery is significant because, for the first time, it allows unlimited quantities of pseudopterosins to be made in a controllable, standardized fashion without requiring collection from environmentally sensitive coral reefs. Pseudopterosin production from a single clone of well-characterized bacteria that can be cultured from frozen stock in perpetuity allows efficient and standardized production methods to be developed. For example, fermentation could be used to produce large quantities of pseudopterosins without the limitations associated with collection from nature (e.g., limited supply, laws banning coral harvesting). For pseudopterosin-based drugs (and other bio-products), such standardized methods should facilitate compliance with manufacturing regulations such as the current good manufacturing practice regulations set forth in title 21, part 211 of the United States' Code of Federal Regulations.

A single bacterium may also be subjected to improvements through mutation or genetic modification, thusly improving the production yield. These same modification methods may also be applied to adjust the ratios of the individual pseudopterosins being produced to better suit a commercial market. Seco-pseudopterosins are intermediates in the biosynthesis of pseudopterosins. These molecules have shown anti-inflammatory activities superior to pseudopterosins, but are found in low abundance in P. elisabethae. A simple mutation resulting in the truncation of the pseudopterosin pathway could produce a bacterial culture capable of producing seco-pseudopterosins. Individual strains can be obtained for all 26 known pseudopterosins derived from a broad geographic distribution, whereas commercial harvesting of wild P. elisabethae is only presently permitted in a single location.

Mixed populations of bacteria are known to be in a constant state of flux as the various members of the population try to out compete others for limited nutrients. This results in meta-stable populations of bacteria where the member of the population responsible for production of a secondary metabolite can be out competed resulting in a loss of production. Due to the inherent instability of mixed cultures, they are often affected by changes in their environment too subtle to effectively control. None of the production controls normally used for fermentative production apply to mixed cultures such as ensuring strain purity and identity.

Accordingly, the invention features a pseudopterosin-producing bacterial culture that includes the culture of an isolated pseudopterosin-producing clonal bacterial strain in a culture medium (e.g., one including nutrients and seawater) that supports the growth of the bacterial strain. The strain can be one isolatable from P. elisabethae. It can also be a Pseudomonas species. The bacterial culture can include at least one pseudopterosin produced by the bacterial strain at a concentration of greater than about 5 micrograms per liter (e.g., greater than about 5 milligrams per liter). It can further include an agent for inducing mutations in the bacterial strain.

In another aspect, the invention features an isolated pseudopterosin-producing clonal bacterial strain. The isolated strain may be frozen, e.g., for preservation and/or use as stock to seed future cultures.

Also within the invention is a bacterial strain library that includes at least a first isolated pseudopterosin-producing clonal bacterial strain and a second isolated pseudopterosin-producing clonal bacterial strain differing from the first strain. In one variation of the library, the first strain produces a first pseudopterosin and the second strain produces a second pseudopterosin having a different chemical structure than the first pseudopterosin.

The invention also features a method of producing a pseudopterosin. The method can include the steps of: providing at least one culture of an isolated pseudopterosin-producing clonal bacterial strain; inoculating a medium with the culture; placing the inoculated medium under conditions that promote production of the pseudopterosin by the bacterium; and purifying the pseudopterosin from the medium. The step of providing at least one culture of an isolated pseudopterosin-producing clonal bacterial strain can include thawing a frozen sample of the isolated pseudopterosin-producing clonal bacterial strain. The step of placing the inoculated medium under conditions that promote production of the pseudopterosin by the bacterium can include incubating the inoculated medium at about 30° C. and/or incubating the inoculated medium for at least 22 days. The step of purifying the pseudopterosin from the medium comprises a step of extracting at least a portion of the medium with an organic solvent to yield an extract comprising the pseudopterosin and, optionally, a step of subjecting the extract to a chromatographic separation.

In yet another aspect, the invention features a method of producing a mixture of at least a first pseudopterosin and a second pseudopterosin, the first pseudopterosin having a different chemical structure than the second pseudopterosin, and the mixture including the first pseudopterosin and a second pseudopterosin in a predetermined molar ratio. This method can include the steps of: purifying the first pseudopterosin from a first bacterial culture including the first pseudopterosin but not the second pseudopterosin; purifying the second pseudopterosin from a second bacterial culture including the second pseudopterosin but not the first pseudopterosin; and mixing the first purified pseudopterosin with then second pseudopterosin in the predetermined ratio.

Unless otherwise defined, all technical terms used herein have the same meaning as commonly understood by one of ordinary skill in the art to which this invention belongs. Definitions of molecular biology terms can be found, for example, in Rieger et al., Glossary of Genetics: Classical and Molecular, 5th edition, Springer-Verlag: New York, 1991; and Lewin, Genes V, Oxford University Press: New York, 1994. Definitions of organic chemistry and enzymology can be found, for example, in R. B. Silverman et al., The Organic Chemistry of Enzyme-Catalyzed Reactions, Academic Press: San Diego, Calif., 2000; and R. T. Morrisson et al., Organic Chemistry, 6th edition, Addison-Wesley Publishing Co.: Boston, Mass., 1992.

As used herein, the phrase “clonal bacterial strain” refers to (i) a single bacterial cell having a first genotype or (ii) a population of cells derived from that single bacterial cell and having the first genotype. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

FIG. 1 is a graph of the results of a UHPLC-MS of PS137 culture extract. A) Extracted ion chromatogram of 445.2 m/z. B) MS³ mass spectrum resulting from the fragmentation of the 299.2 m/z ion resulting from the fragmentation of 445.2 m/z. Three spectra are averaged over the range 4.97-5.03 minutes.

FIG. 2 is a graph of the results of UHPLC-MS of authenticated pseudopterosin G. A) Extracted ion chromatogram of 445.2 m/z. B) MS³ mass spectrum resulting from the fragmentation of the 299.2 m/z ion resulting from the fragmentation of 445.2 m/z. Three spectra are averaged over the range 4.93-4.99 minutes.

FIG. 3 is a graph showing pseudopterosin G content of cultures at 3 day intervals in three replicate cultures of PS 137.

### DETAILED DESCRIPTION

The invention encompasses isolated pseudopterosin-producing bacterial strains, libraries of such strains, cultures of such strains, and method for producing a pseudopterosin or mixture of pseudopterosins without the massive destruction of coral reefs. The below described preferred embodiments illustrate adaptations of these strains, libraries, cultures and methods. Nonetheless, from the description of these embodiments, other aspects of the invention can be made and/or practiced based on the description provided below.

Pseudopterosin-producing Bacterial Strains. Bacteria useful in the invention can be any that produces a pseudopterosin. As described herein suitable such bacteria can be isolated from the gorgonian P. elisabethae, a purple frilly seafan, commonly found in the shallow-water reefs of the tropical Atlantic including regions of the Caribbean including near Bimini in The Bahamas. For example, live samples of P. elisabethae can be harvested from the environment and then processed to collect and expand the pseudopterosin bacteria present therein. In an exemplary protocol, live P. elisabethae specimens are cut into smaller pieces and homogenized in a blender. Large coral pieces can be removed by coarse filtration, and the bacteria-containing filtrate can be repeatedly washed and centrifuged. The resulting pellet can then be separated by density centrifugation (e.g., using discontinuous Percoll® gradients and collecting the band of material at the interface between 30% and 70% Percoll®. This zooxanthellae-enriched fraction can be cultured in medium that supports the growth of the bacteria therein (e.g., in Nutrient Broth [NB] medium made with seawater) at about 37° C. (e.g., between about 25-40° C. such as at 24, 25, 26, 27, 28, 29, 30, 31, 32, 33, 34, 35, 36, 37, 38, 39, 40, and 41° C.) in loosely capped culture flasks under ambient conditions without shaking. The cultures can be repeatedly subcultured and frozen (e.g., in glycerol or DMSO at −80° C. or colder) at any stage. Clonal bacterial strains can be isolated from these mixed cultures by streaking an aliquot of a culture on solid bacterial growth medium and then picking the individual bacterial colonies that result. These isolated clonal bacterial strains can be used to inoculate sterile liquid bacterial growth medium to make cultures of the individual isolated clonal bacterial strains. Each of the cultures can be analyzed for the presence of one or more pseudopterosins (or synthetic intermediaries thereof; see U.S. Pat. No. 6,780,622) to identify those strains that produce one or more pseudopterosins (or synthetic intermediaries thereof). Isolated pseudopterosin-producing cells might also be used to make other cells that produce a pseudopterosin or synthetic intermediary thereof. See, e.g., Zhang et al., Molecular Pharmaceutics, Vol. 5, pp. 212-225; and Malpartida and Hopwood 1984, Nature, Vol. 309, pp. 462-464.

For example, a sample of an isolated pseudopterosin-producing clonal bacterial strain can be exposed to a mutagen such as ethyl methane sulfonate or nitrosoguandine to induce random mutations in the strains genomic DNA. Individual bacteria in the sample can be isolated by streaking and picking of individual colonies. The resulting individual colonies can be cultured and tested for pseudopterosin production. Those colonies displaying a desirable characteristic (e.g., producing high levels of pseudopterosin, producing a particular pseudopterosin, derivative thereof, synthetic intermediary thereof [such a seco-pseudopterosin], or mixtures of the foregoing) can be selected for further use.

Libraries. Two or more (e.g., 2, 3, 4, 5, 6, 7, 8, 9, 10, 20, 30, 50, 100 or more) different pseudopterosin-producing strains can be combined to form a library of different strains having different characteristics (e.g., a first strain produces a first pseudopterosin or mix of pseudopterosins, a second strain produces a second pseudopterosin or mix of pseudopterosins differing from the first, and a third strain produces a third pseudopterosin or mix of pseudopterosins differing from the first and second). A preferred library is one that includes at least 26 different strains, wherein each of the strains produces a different pseudopterosin such that the library can be used to produce the 26 known types of pseudopterosin for convenient use in screening assays. The two or more different strains can be stored in separate vials, e.g., in a −80° C. freezer or in liquid nitrogen. Alternatively, a single container with multiple wells or storage units that each hold a single strain can be used.

Pseudopterosin-producing Bacterial Cultures. One or more (e.g., 1, 2, 3, 4, 5, 6, 7, 8, 9, 10, 20, or more) pseudopterosin-producing bacterial strains can be mixed with a medium that supports its growth to form a pseudopterosin-producing bacterial culture. Any suitable medium might be used. In the examples, described below nutrient broth (3 g beef extract and 5 g peptone pr liter; “NB”) in seawater was used. The culture can be placed under any suitable conditions that promote the growth of the bacteria and/or production of pseudopterosin. (e.g., under ambient atmospheric conditions; at about 25, 26, 27, 28, 29, 30, 31, 32, 33, 34, 35, 36, 37, 38, 39, or 40° C.; in a culture flask, without shaking). Other factors such as one or more quorum sensing molecules, host factors (i.e., agents produced by P. elisabethae that modulate pseudopterosin production by a bacterium), and other factors that enhance terpene production (e.g., plant growth factors such as methyl salicylate) might be added to a culture. Selectable markers such as a nucleic acid that encodes antibiotic resistance might be introduced into a strain of pseudopterosin-producing bacteria, e.g., to prevent contamination of pure cultures.

Method for Producing A Pseudopterosin And/or Synthetic Intermediaries Thereof. One or more pseudopterosins (or synthetic intermediaries thereof) can be made by placing a pseudopterosin-producing culture of bacteria under conditions that promote growth of the bacteria and/or production of one or more pseudopterosins. Pseudopterosins and/or synthetic intermediaries thereof such as seco-pseudopterosins can be purified from the cultures by adapting known procedures such as those described by Look et al., Proc. Natl. Acad. Sci. USA. 83:6238-6240, 1986; Look et al., J. Org. Chem. 51:5140-5145, 1986; Look et al., Tetrahedron 43:3363-3370, 1987; Roussis et al., J. Org. Chem. 55:4916-4922, 1990; and U.S. Pat. Nos. 4,849,410, 4,745,104, and 5,624,911. In addition, for high recovery (e.g., greater than about 90%) pseudopterosins can be purified from cultures using a resin such as HP20, Amberlite XAD2, XAD7, XAD1180, or C-18. For example, HP20 resin is added to a culture of pseudopterosin-producing bacteria (e.g., at a ratio of 1 mL resin/5 mL culture) and mixed (e.g., for at least about 30 minutes). The resin is then filtered and washed with water and then methanol. The methanolic fraction is then fractionated over a C-18 cartridge prior to purification by HPLC (or analysis by UHPLCMS). Pseudopterosin-containing products can contain different amounts of pseudopterosins as desired for a particular application. For example, a product might contain about 0.001-100% pseudopterosin by weight (e.g., 0.0009, 0.001, 0.01, 0.1, 1, 2, 3, 4, 5, 6, 7, 8, 9, 10, 20, 30, 40, 50, 60, 70, 80, 90, 91, 92, 93, 94, 95, 96, 97, 98, 99, 99.5, 99.9, 99.99, or 99.999% pseudopterosin by weight). Pharmaceutical grade pseudopterosins will be sterile and lack significant amounts of pyrogens.

Method for Producing A Mixture Of Pseudopterosins. Once produced various purified pseudopterosins can be mixed together to yield a desired product. For example, at least a first pseudopterosin and a second pseudopterosin (could be, e.g., 2, 3, 4, 5, 6, 7, 8, 9, 10, 11, 12, 13, 14 15, 16, 17, 18, 19, 20, 21, 22, 23, 24, 25, or 26 different pseudopterosins), wherein the first pseudopterosin has a different chemical structure than the second pseudopterosin, can be mixed together in a predetermined molar ratio (e.g., 1:1, 1:2, 1:3, 1:4, 1:5, 1:6, 1:7, 1:8, 1:9, 1:10, 1:20, 1:50, 1:100, 1:250, 1:500, or 1:1000) to make the desired product. This method can include the steps of: purifying the first pseudopterosin from a first bacterial culture including the first pseudopterosin but not the second pseudopterosin; purifying the second pseudopterosin from a second bacterial culture including the second pseudopterosin but not the first pseudopterosin; and mixing the first purified pseudopterosin with then second pseudopterosin in the predetermined ratio.

### EXAMPLES

The present invention is further illustrated by the following specific examples. The examples are provided for illustration only and are not to be construed as limiting the scope or content of the invention in any way.

### Example 1

### Isolation of Clonal Strains of Bacteria that Produce Pseudopterosins

Materials & Methods

Media and chemicals used: except where otherwise specified, all media and chemicals were purchased from Fisher Scientific. All fresh water was double deionized “Nanopure” water. Sea water was obtained from the Gumbo Limbo Environmental Complex (Boca Raton, Fla.), and was filtered through a 0.22 μm sterile filter prior to use. Artificial sea water was prepared by adding 36 g of Instant Ocean® brand synthetic sea salts per liter of water followed by sterilization by autoclaving. Nutrient broth (NB) medium was prepared by adding 5 g peptone and 3 g meat extract per liter of water followed by sterilization by autoclaving. NB in sea water medium was prepared by adding 36 g Instant Ocean®, 5 g peptone, and 3 g meat extract per liter of water followed by sterilization by autoclaving. Solid agar media were prepared by the addition of 10 g agar per liter of water to the previously described liquid formulations.

Pseudopterosin assay: HP20 resin (5 mL was added to 25 mL of culture and the sample agitated for 30 mins at 150 rpm. The resin was then filtered and washed with water (15 mL) and methanol (15 mL). The methanol extract was fractionated over a C-18 column into four fractions: 1) H₂O, 2) H₂O:MeOH (1:1), 3) MeOH, and 4) CH₂Cl₂. The third fraction was evaporated, dissolved in 100 uL methanol and 20 uL was analyzed by LC-MS.

Other resins such as Amberlite XAD2, XAD7, XAD1180, C-18 and other purification strategies such as lyophilization followed by extraction can also be used to purify pseudopterosins from cultures.

Analysis for pseudopterosin content: samples were analyzed on a Thermo Scientific Accela-LXQ UHPLC-MS fitted with a Hypersil Gold C-18 column (50 mm×2.1 mm, 1.9 μm particle size). The sample size injected was 1.5 μL. The mobile phase was a gradient of water and methanol at 400 μL per minute. The gradient was programmed as follows: elution with 50% water 50% methanol, 1 minute; gradient to 100% methanol, 4 minutes; 100% methanol, 5 minutes; and reequilibration with 50% methanol 50% water, 1 minute. The eluate was monitored by an Accela PDA detector scanning 200-800 nm and monitoring 229 nm, 276 nm, and 286 nm. The eluate was also analyzed by a LXQ ion trap mass spectrometer performing 6 sequential scan events in negative ion mode as follows: Scan event 1: Scan 50.0-800.0 m/z; Scan event 2: MS2 of 445.2 m/z, scanning 150.0-500.0 m/z; Scan event 2: MS3 of the 299.2 m/z fragment of 445.2 m/z, scanning 80.0-300.0 m/z; Scan event 4: MS2 of 487.2 m/z, scanning 130.0-500.0 m/z; Scan event 5: MS3 of the 445.2 m/z fragment of 487.2 m/z, scanning 150.0-500.0 m/z; Scan event 6: MS4 of the 299.2 m/z fragment of 445.2 m/z fragment of 487.2, scanning 80.0-300.0 m/z.

Extraction of authenticated pseudopterosins: live P. elisabethae specimens were collected by hand using SCUBA in the waters near Bimini, The Bahamas. The specimens were dried in the sun and stored at room temperature pending extraction. The dry P. elisabethae was extracted sequentially with 600 mL each of ethyl acetate, methylene chloride, and 50:50 ethyl acetate:methylene chloride. These extracts were combined and the solvents were evaporated under reduced pressure, yielding the crude extract. The crude material was dissolved in methanol/water (9:1) and portioned with hexanes to give a nonpolar extract. The ratio of the methanol water was adjusted to 1:1, and the aqueous layer was portioned with methylene chloride. The methylene chloride partition served as a standard mix of pseudopterosins G, H, I, & J in screening experiments.

Pseudopterosin G was purified from the methylene chloride partition by preparative TLC on using 50:50 ethyl acetate:hexane mobile phase. Individual bands were visualized by UV, and the corresponding region of silica was removed with a razor blade. Pseudopterosin G were extracted with ethyl acetate and purified by HPLC. The identity of the pseudopterosin G was confirmed by NMR.

Strain isolation: live P. elisabethae specimens were collected by hand using SCUBA in the waters near Bimini, The Bahamas and held in aquaria. A zooxanthellae-enriched fraction was obtained from a single live coral specimen. Approximately 10 g of P. elisabethae was clipped into ˜1 cm pieces with scissors. The pieces of coral were washed in 50% sea water diluted with fresh water. Coral pieces were removed from transferred to a sterile blender with approximately 25 mL 50% sea water. The coral was blended using short bursts at maximum power. The blended coral were filtered through 4 layers of sterile cheesecloth to remove large coral fragments. The filter cake was rinsed once with 15 mL of 50% sea water. The filtrate was centrifuged at 370×g for 3 minutes, the supernatant was discarded, and the pellet was resuspended in 50 mL of 50% sea water. The pellet was centrifuged and resuspended in this same manner 10 times. The washed pellet was stored overnight at 4° C. The washed pellet was further enriched for zooxanthellae by buoyant density centrifugation using discontinuous Percoll® gradients. Percoll® gradients were prepared by layering 10 mL of 30% Percoll® in 50% sea water over 7.5 mL of 70% Percoll®. To these prepared gradients was overlaid the washed pellet followed by centrifugation at 10⁵×g for 10 minutes. The band of material at the interface between 30% and 70% Percoll was collected, diluted to 50 mL with 50% sea water, and pelleted at 370×g for 5 minutes. The pellet was resuspended in 20 mL 50% sea water and stored at 4° C.

To 250 mL NB medium in a 500 mL flask was added the zooxanthellae-enriched fraction. This culture (PE8) was grown without shaking at 37° C. in loosely capped culture flasks under ambient conditions. After 2 days, 40 mL of this culture was used to inoculate 400 mL of NB medium. This culture was grown at 37° C. without shaking for 134 days. Two and one half milliliters of this culture was inoculated into 250 mL NB. This culture (PE8-sub1) was grown without shaking at 30° C. for 222 days. An aliquot of this culture (PE8-sub2) was mixed with glycerol to a final concentration of 30% glycerol and held frozen at −80° C.

Ten milliliters of NB medium in sea water was inoculated with a small (˜5 μL) piece of frozen PE8-sub2 freezer stock. After 3 days at 30° C. without shaking, 1.5 mL of the 10 mL culture was inoculated into 150 mL of NB in sea water (PS10). This culture incubated at 30° C. for 29 days without shaking. The product of this culture was diluted 1 in 10,000 in NB in seawater medium, then 100 μL of diluted culture was plated on solid NB in sea water agar plates. Plates were incubated for 2 days at 30° C. Individual colonies were picked and used to separately inoculate 45 mL aliquots of NB in sea water (cultures PS116 through PS155). After 14 days at 30° C., the cultures were screened for pseudopterosins by UHPLC-MS as previously described and glycerol stocks were placed at −80° C.

Fourteen different cultures showed pseudopterosin production in initial screening. Single cultures were streaked from glycerol stocks onto solid agar NB in sea water medium. Single colonies were serially streaked 4 times to ensure strain purity. A single colony from the cultures inoculated 50 mL of NB in sea water. After 2 days at 30° C., this culture inoculated culture tubes each containing 45 mL of NB in sea water with 450 μL of inoculum. After 8 days at 30° C. without shaking, the contents of the tubes were assayed for pseudopterosin production as previously described.

16S isolation: 16S rDNA was amplified from genomic DNA of Pseudomonas sp. strain PS137. gDNA from pelleted Pseudomonas sp. strain PS137 from 10 mL of culture following 2 days in NB in sea water at 30° C. without shaking was purified using the Qiagen Genomic Tip 100/G kit according to the manufacturer's instructions for bacteria. The 16D rDNA was amplified by polymerase chain reaction (PCR) in a 50 μL reaction containing 1× thermostable polymerase buffer (20 mM Tris-HCl pH 8.8, 2 mM MgSO4 10 mM KCl, 10 mM (NH4)2SO4, 0.1% Triton X100), 0.025 mM of each dNTP, 1 μM each of primers RC1492 (TAC GGY TAC CTT OTT ACG ACT T) (SEQ ID NO:2) and 16FC27 (AGA GTT TGA TCC TGG CTC AG) (SEQ ID NO:3), 1-2 ng gDNA, and 2.5 U Taq polymerase (NEB). The PCR program was 95° C. for 1 min followed by 30 cycles of 95° C. for 45 sec, 55° C. for 45 sec, and 72° for 1 min followed by 72° C. for 3 min. The ˜600 bp PCR product was gel purified (Qiagen) and sequenced (Analytical Genetics Technology Centre, Toronto, ON). Sequences were analyzed using the blastn algorithm.

Results

A zooxanthellae-enriched fraction was isolated from approximately 10 g of P. elisabethae collected from the waters near Bimini, The Bahamas. This fraction was inoculated into culture medium to produce a mixed bacterial assemblage from the bacteria closely associated with the zooxanthellae. After several subcultures, the resulting bacterial assemblage was diluted and plated on solid agar medium. From the bacterial colonies that grew on the solid medium, 40 colonies were screened for the production of pseudopterosins.

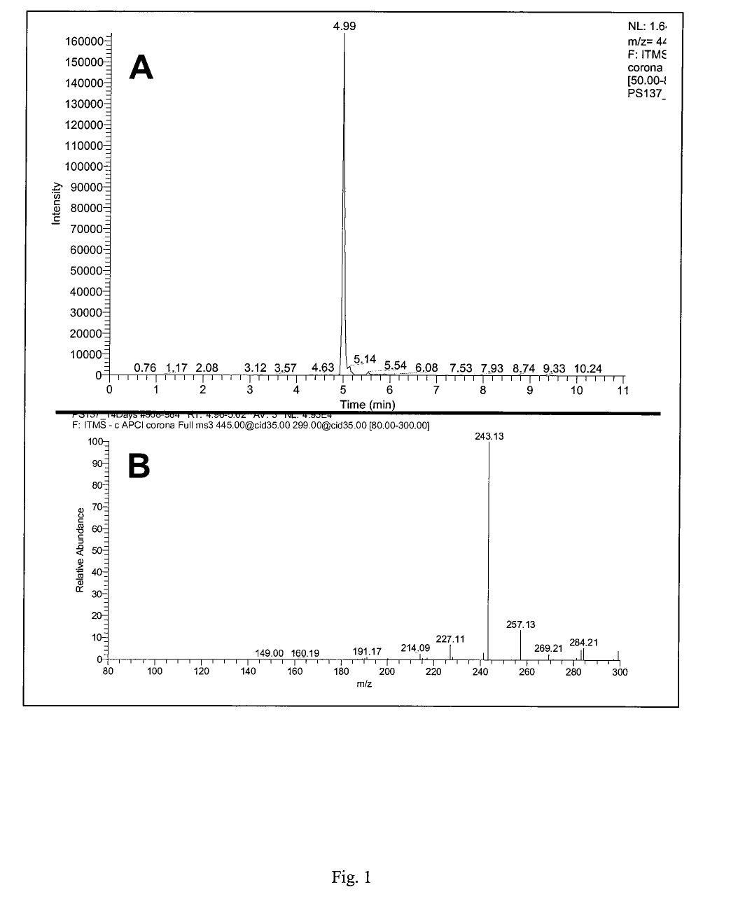

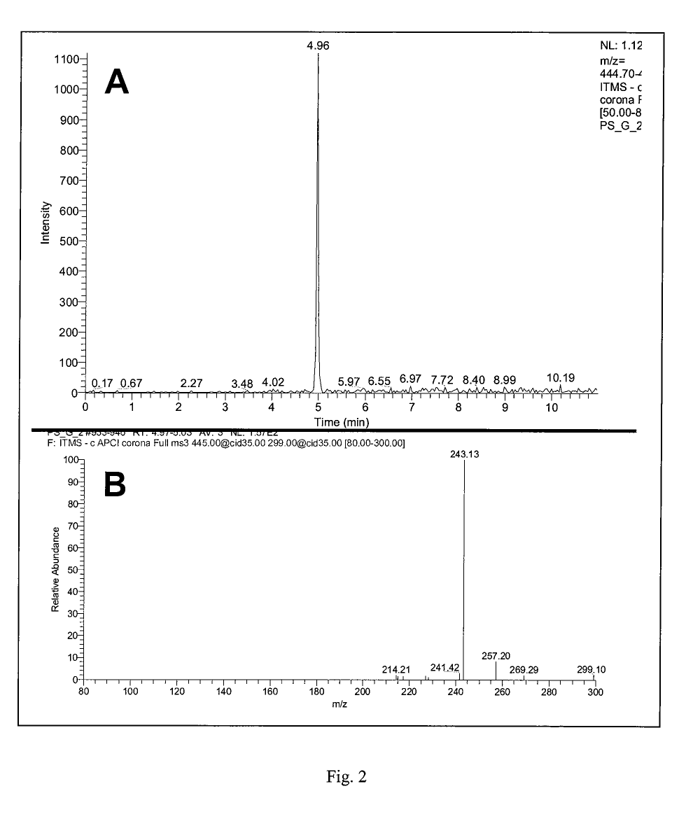

Several cultures were shown to produce pseudopterosins G and H-J at various ratios. FIG. 1 shows the extracted ion chromatogram of 445.2 m/z for one such strain, PS137. The MS3 of the peak at R.T. 5.00±0.03 minutes has been shown to match that of authenticated pseudopterosin G at the same retention time (FIG. 2). Peaks at R.T. 5.03±0.03 minutes and 5.19±0.01 minutes with molecular ion 487.2 m/z were also seen. These peaks are consistent with those of the acetylated pseudopterosins H, I, & J. Methylene chloride extracts of Bimini P. elisabethae contained identical peaks to the 487 m/z peaks found in the culture extracts. Extracts from culture samples taken at the time of inoculation contained no detectable pseudopterosins. All LCMS data for the compounds produced by Pseudomonas sp. strain PS137 were identical in all respects with that from authenticated standards of Ps G, H, I and J. The HPLC retention times (RT) were identical and the MS data indicated the presence of identical molecular ions, MS² and MS³ spectra.

The growth and analysis was repeated for culture PS137. The HPLC-MS chromatogram and spectra obtained were identical to those described in FIG. 1. 16S rDNA was amplified from five single colonies of culture PS137 by PCR and sequenced. SEQ ID NO:1 below is the consensus sequence of the five individual sequences. This sequence was identified by the BLAST algorithm as originating from a species of the genus Pseudomonas.

### Example 2

### Time Course Study to Demonstrate Production of Pseudopterosin G in PS137

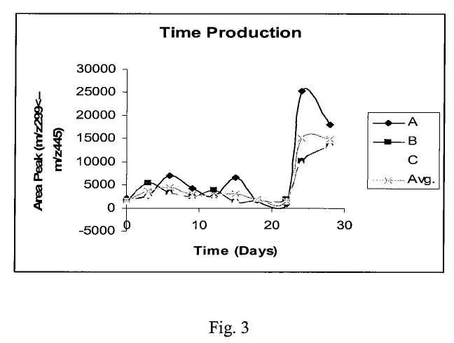

Aliquots of three replicate cultures (as described in Example 1) were taken at three day intervals and each analyzed by LCMS for pseudopterosin G (PsG) content. As shown in FIG. 3, there was a dramatic increase in PsG content at day 22 in all three replicates.

### Other Embodiments

It is to be understood that while the invention has been described in conjunction with the detailed description thereof, the foregoing description is intended to illustrate and not limit the scope of the invention. Other aspects, advantages, and modifications are within the scope of the following claims.

What is claimed is:

1. A pseudopterosin-producing bacterial culture, the culture comprising an isolated pseudopterosin-producing clonal bacterial strain in a culture medium that supports the growth of the bacterial strain.

2. The culture of claim 1, wherein the strain is isolatable from Pseudopterogorgia elisabethae.

3. The culture of claim 1, wherein the strain is a Pseudomonas species.

4. The culture of claim 4, wherein the strain comprises a nucleic acid comprising the sequence of SEQ ID NO:1.

5. The culture of claim 1, wherein the culture comprises at least one pseudopterosin produced by the bacterial strain at a concentration of greater than 5 micrograms per liter.

6. The culture of claim 1, wherein the culture comprises at least one pseudopterosin produced by the bacterial strain at a concentration of greater than 5 milligrams grams per liter.

7. The culture of claim 1, wherein the culture consists essentially of the isolated pseudopterosin-producing clonal bacterial strain and the culture medium.

8. The culture of claim 1, further comprising an agent for inducing mutations in the bacterial strain.

9. The culture of claim 1, wherein the medium comprises nutrients and seawater.

10. An isolated pseudopterosin-producing clonal bacterial strain.

11. The bacterial strain of claim 8, wherein the strain is frozen.

12. A bacterial strain library, the library comprising at least a first isolated pseudopterosin-producing clonal bacterial strain and a second isolated pseudopterosin-producing clonal bacterial strain, the first strain differing from the second strain.

13. The library of claim 12, wherein the first strain produces a first pseudopterosin and the second strain produces a second pseudopterosin, the first pseudopterosin having a different chemical structure than the second pseudopterosin.

14. A method of producing a pseudopterosin, the method comprising the steps of:
providing at least one culture of an isolated pseudopterosin-producing clonal bacterial strain;
inoculating a medium with the culture;
placing the inoculated medium under conditions that promote production of the pseudopterosin by the bacterium; and
purifying the pseudopterosin from the medium.

15. The method of claim 14, wherein the step of providing at least one culture of an isolated pseudopterosin-producing clonal bacterial strain comprises thawing a frozen sample of the isolated pseudopterosin-producing clonal bacterial strain.

16. The method of claim 14, wherein the medium comprises nutrients and seawater.

17. The method of claim 14, wherein the step of placing the inoculated medium under conditions that promote production of the pseudopterosin by the bacterium comprises incubating the inoculated medium at about 30° C.

18. The method of claim 14, wherein the step of placing the inoculated medium under conditions that promote production of the pseudopterosin by the bacterium comprises incubating the inoculated medium for at least 14 days.

19. The method of claim 14, wherein the step of purifying the pseudopterosin from the medium comprises a step of extracting at least a portion of the medium with an organic solvent to yield an extract comprising the pseudopterosin.

20. The method of claim 19, wherein the step of purifying the pseudopterosin from the medium further comprises a step of subjecting the extract to a chromatographic separation.

21. A method of producing a mixture of at least a first pseudopterosin and a second pseudopterosin, the first pseudopterosin having a different chemical structure than the second pseudopterosin, and the mixture comprising the first pseudopterosin and a second pseudopterosin in a predetermined molar ratio, the method comprising the steps of:
purifying the first pseudopterosin from a first bacterial culture comprising the first pseudopterosin but not the second pseudopterosin;
purifying the second pseudopterosin from a second bacterial culture comprising the second pseudopterosin but not the first pseudopterosin; and
mixing the first purified pseudopterosin with the second pseudopterosin in the predetermined ratio.


SEQUENCE LISTING (3 sequences)

SEQ ID NO: 1 - DNA, 1391 bp, Pseudomonas sp.
tgcagtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaatccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaaaccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccgcaa

SEQ ID NO: 2 - DNA, 22 bp, artificial sequence
  primer
tacggytaccttgttacgactt

SEQ ID NO: 3 - DNA, 20 bp, Artificial sequence
  primer
agagtttgatcctggctcag